(19) United States
(12) Patent Application Publication
(10) Pub. No.: US 2026/0224354 A1
(43) Pub. Date: Aug. 6, 2026

(54) Improvements for Balloon Expandable (BE) and Self Expandable (SE) Heart Valves and Heart Valve Delivery Devices

(71) Applicant: Foldax, Inc., Salt Lake City, UT (US)

(72) Inventors: Megan Nguyen, Salt Lake City, UT (US); James G. Wittel, Salt Lake City, UT (US); Garrett Johnson, Costa Mesa, CA (US); Praveen De Silva, Irvine, CA (US); Marek Lhotak, Torrance, CA (US)

(21) Appl. No.: 19/159,589

(22) PCT Filed: Feb. 26, 2024
(86) PCT No.: PCT/US2024/017352

§ 371 Date: Aug. 25, 2025

Related U.S. Application Data
(60) Provisional application No. 63/448,192, filed on Feb. 24, 2023.
(60) Provisional application No. 63/545,220, filed on Oct. 23, 2023.

(51) Int. Cl. A61F 2/24

(52) U.S. Cl. CPC A61F 2/2415; A61F 2/2418; A61F 2/2433; A61F 2210/0076; A61F 2240/001; A61F 2250/0097

(57) ABSTRACT

The invention includes improvements to the design and structure of prosthetic cardiac valves having a polymeric component and particularly the leaflet structure formed by polymers integrated with an expandable and collapsible structural support. Improved manufacturing techniques are also provided to enable improved designs and performance of a prosthetic valve. Similarly, improvements to delivery systems for the particular valve designs described herein are also provided. The improvements include both balloon expanded and self-expanded prosthetic valve structures and improvements to each of the prosthetic valve, the components of the manufacturing process and the delivery system.

### BACKGROUND OF THE INVENTION

Disease of the heart valves is an important cause of cardiovascular dysfunction and death. When a patient suffers from valvular heart disease, one treatment is surgical replacement of a diseased or dysfunctional valve with a prosthetic valve. The first cases where prosthetic cardiac valves were used to replace a patient's diseased or dysfunctional valve used open-chest surgery to allow external access to the heart and the ability to surgically place a replacement prosthetic cardiac valve at the target site of the diseased native valve. To avoid the need for open-chest surgery, physicians and engineers developed minimally invasive techniques and devices where the new prosthetic valve was able to be collapsed into a very narrow diameter, confined inside the end of a catheter and introduced at a peripheral point in the patient's vasculature such as an artery inside the thigh. Using the minimally invasive catheter, the prosthetic valve is advanced through the patient's circulatory system and until reaching to the location where the prosthetic valve replacement is needed.

Prosthetic valves suitable for the minimally invasive approach must have some unique design and performance features such as the ability to be radially collapsed down to the small diameter inside the delivery catheter used to introduce the prosthetic valve to the patient's vasculature and then be able to be expanded at the target site in the patient's heart. Once proper placement of the prosthetic valve in the collapsed state is achieved, some valves are designed to self-expand and other prosthetic valves are expanded using a balloon incorporated into the delivery catheter. The ability to implant of prosthetic heart valves through either a balloon-expandable delivery system or a system designed for self-expandable valves played a major role in the early stages of this minimally invasive technique, known in the industry as transcatheter aortic valve replacement (TAVR).

The TAVR technology thereby facilitates placement of a collapsible prosthetic cardiac valve in a minimally invasive fashion to replace the diseased native valve. The collapsible prosthetic valves typically contain movable leaflets that form the portion of the valve that opens and closes in response to blood pressure pulses occurring across the valve. The leaflet portion of the prosthetic valve is formed inside a metallic stent structural frame that is itself collapsed to a size less than the internal diameter of the delivery catheter and expandable to a predetermined diameter at patient's native heart annulus. In practice, the prosthetic valve is manufactured to the expanded configuration and then later crimped into a size smaller than the internal diameter of the delivery catheter. In balloon-based delivery systems, the prosthetic valve may be configured to surround, or be placed immediately proximate to, an inflatable balloon that is expanded inside the heart to cause the prosthetic valve to engage the patient's heart at the target site. Other valve designs are designed to be self-expanding such that the process of releasing the valve from the delivery catheter causes the valve to expand in place. In either case, either self-expansion of the valve or balloon inflation, the valves and delivery systems must have special design features to take advantage of the minimally invasive TAVR.

The use of self-expanding and balloon-expandable valves in the TAVR approach underwent a rapid expansion in the years following the original pioneering work and a series of modifications to both the prosthetic valves and the delivery systems have been introduced over the last 2 decades as the TAVR procedure has become more widely adopted and as the safety of the TAVR procedure has been established in large multicenter clinical trials that have confirmed the safety and efficacy of TAVR using both self-and balloon-expandable valves. However, many challenges in developing prosthetic valves for the TAVR procedure remain because the valve design itself must provide years to decades of proper function inside the patient's heart without structural or mechanical failure and without undergoing chemical or biochemical degradation over billions of cycles of opening and closing inside the beating heart over decades of use following implantation in the patient. This capability to continue to function continuously over many years and billions of cycles also requires a delivery system that places the prosthetic valve at the native annulus with significant precision such that deployment of the prosthetic valve from the distal end of a delivery catheter is able to be carefully controlled and in a manner that allows a surgeon to verify in real time that the prosthetic valve has been properly placed. For this reason, there is an ever present need to develop improved designs for prosthetic cardiac valves, their constituent materials, and delivery systems for minimally invasive cardiac valve replacement procedures.

Current prosthetic valve replacement options may also be limited by structural valve degeneration requiring reoperation to replace or repair the original prosthetic valve or may create the need for lifelong treatment with anticoagulation drugs. In addition to simple mechanical failure, many existing designs incorporate animal tissue as part of the valve leaflets and these animal tissue materials may be reactive with human blood and may undergo calcification or formation of clots or thrombus that threaten their long-term efficacy following implantation. To avoid some of these drawbacks, new polymer technologies have been developed in recent years in the hope of creating an ideal polymeric heart valve substitute that overcomes these limitations. Polymeric materials and prosthetic valves using these materials are described in US Patents and Publications U.S. Pat. Nos. 9,301,837, 9,539,089, 10,918,477, 10,213,833, 10,286,657, 10,723,844, 11,534,293, 11,129,712, US Publ. 20190060061A1, U.S. Pat. No. 11,000,369, US 20210038379A1 that are specifically incorporated herein by reference. These compounds and prosthetic valve devices are in various stages of research and development and have both unique advantages and design and manufacturing challenges inherent to the use of polymer and the properties of a prosthetic valve incorporating polymers into the overall design of the valve itself and the delivery system.

Accordingly, research and development of improved valve structures, delivery systems, and manufacturing methods will continue to improve the performance of prosthetic cardiac valves for the TAVR approach. In particular, where polymer materials are used, an ongoing need exists for new and inventive designs and configurations for the valve structure itself, particular manufacturing methods using polymeric materials, and improved designs for delivery systems for such prosthetic devices to advance clinical acceptance of such surgical techniques and devices and to improve the health of patients suffering from valvular cardiac disease.

### BRIEF SUMMARY OF THE INVENTION

The invention includes improvements to the design and structure of prosthetic cardiac valves having a polymeric component and particularly a leaflet structure formed by polymers integrated with an expandable and collapsible structural support. Improved manufacturing techniques are also provided to enable improved designs and performance of such a prosthetic valve. Similarly, improvements to delivery systems for the particular valve designs described herein are also provided.

Specifically with respect to prosthetic valve design and structure, the expandable support structure has an open frame design to provide better fluid access to the coronary arteries within the heart and to protect the polymer leaflet structures from impacting against the frame during introduction and deployment. The support structure is comprised of a lattice of individual cells are sized and configured to allow the polymer used to form the leaflets to integrally mold together with the lattice to form a unitary structure that is able to compress during the crimping step where the expanded form configuration of the valve is crimped into the smaller diameter necessary for introduction into the reduced diameter of the delivery catheter. The frame design may be formed from either a polymer or a shape memory metal where both options provide a collapsible structure that has an initial configuration as manufactured and as designed upon deployment after confinement into a collapsed configuration in the delivery catheter. The aspect ratios, height versus width dimensions and ratios, and other dimensional parameters are optimized to reduce the strain induced on the polymer leaflet structures and the polymer incorporated into the individual cells of the lattice support structure.

Secondary support structures are incorporated into a primary support structure to minimize deflections of the commissures, the points at which adjacent polymer leaflets join the support structure, along a length thereof in a predetermined configuration for optimal performance of the expandable support structure and improved cooperation with the polymeric leaflet material. In one aspect, the geometry of an individual strut of the support structure avoids contact with the polymer leaflets during the crimping process. The secondary support strut also isolates the prosthetic polymer leaflets from being damaged when the prosthetic valve is deployed and displaces the native leaflets at the target annulus. In advanced valvular disease, calcified native leaflets in a patient may impact the new polymer leaflets in the prosthetic valve and mechanically damage the valve thereby reducing the long-term viability. The designs disclosed herein anticipate this potential issue and configure the prosthetic valve to avoid being damaged in this fashion.

Furthermore, with respect to the improved design of the prosthetic valve, the prosthetic valve has both an inflow portion where the blood first enters the prosthetic structure, and an outflow portion downstream or distal to the valve structure where blood leaves the lengthwise dimension of the prosthetic valve. Pursuant to the invention, a self-expanding sealing skirt is configured to surround the outflow portion of the prosthetic valve and circumferentially surround the collapsible support structure. The self-expanding sealing skirt can be used with either a self-expanding support frame comprising the polymeric valve leaflets or may be used in combination with a balloon-expandable support frame comprising the polymeric leaflets. The inner wall of the self-expanding sealing skirt is covered with polymer, preventing any metal-on-metal abrasion with the inner leaflet frame. The sealing skirt has a predetermined height at the inflow portion of the support structure and has two distinct geometric configurations. At the end most proximate to the inflow portion, the sealing skirt is substantially annular and conforms to the circumferential portion and shape of the expandable support stent. At the opposite end, most proximate the outflow portion of the prosthetic valve, the sealing skirt has a scalloped or undulating shape wherein portions of the sealing skirt spaced around the outer portion of the annular support structure are not affixed to the outer layer of the support structure. These non-attached portions of the scalloped edge can expand radially outward from the access of the blood flow through the valve and provide an additional sealing structure against leakage passing in the direction of blood flow and around the outer wall of the support structure and provides an additional series of attachment points around the outer portion of the inflow portion of the prosthetic valve.

The structural frame also includes a marker incorporated into the structural frame that is detectable though existing imaging modalities to enable annular alignment and positioning of the prosthetic valve within the native annulus. Although a number of different geometries are possible, the marker may be formed as part of the structural frame as an open cavity between adjacent cells of the frame structure. The marker is placed in a unique location or locations as part of the structural frame so that the image generated by the marker can be used for both annular alignment in the native annulus and positioning relative to the coronary arteries. Although more than one marker can be used, the marker should be symmetric either as a single structural feature or a structural feature that uniquely and readily conveys the position of the structural frame element using an imaging modality.

Another improved embodiment of the prosthetic valve features a two-part valve assembly that includes a separate sealing skirt frame that is deployed separately from the assembly comprising the prosthetic leaflets. In this embodiment, the sealing skirt frame first engages the native annulus and is expanded to retract the existing diseased leaflets and valve structure. Next, a support structure containing the polymer leaflets is deployed to meet with the sealing skirt frame and provide an integrated prosthetic valve assembly. Either or both of the primary sealing skirt frame or the secondary leaflet frame contains structural features for mating engagement of the two components of the assembly. This embodiment enables a smaller total outer diameter in the collapsed configuration of either or both components compared to the one-peace assembly. As described below, this embodiment may feature a two-balloon delivery catheter employed with each of the sealing skirt frame and the polymer leaflet frame disposed about the periphery of two inflatable balloons and incorporated into a single delivery system.

With respect to the improved manufacturing processes, manufacturing methods enable a closed cell self-expandable or balloon expandable heart valve frame that is dipped cast in polymer without the need for an open cell structure or lattice in the support frame. In this process, polymer leaflets are formed without integral connection with the support frame. The leaflets are cured, measured, and trimmed independently and prior to attachment to the structural frame. Subsequently, leaflet and frame masks are attached to the frame and leaflet assembly to prepare for a subsequent dipping step where polymer flows around unmasked struts of the structural frame and causes the selected unmasked portion of the leaflets to bond to structural side wall using the dipped polymer. Following the step, the leaflet in structural frame assembly is placed in an environmental humidity chamber to complete formation of the assembly under specified conditions. Following exposure to the environmental humidity chamber conditions the leaflet and frame it masking is removed, and the assembly subsequently cured to produce the final prosthetic valve construct and assembly. See US Publ. 20190060061A1 that is specifically incorporated by reference herein.

In another improved manufacturing process embodiment, the sealing skirt is formed on the structural frame by electrospinning directly on the frame. The skirt and structural frame are joined with a separate polymer leaflet structure using leaflet masks to attach the cured polymer leaflet structure to the expandable frame and in the same process completing the outflow edge seal over the sealing skirt. The valve assembly is completed by sealing the inflow edge of the base of the frame to both the frame and the sealing skirt and completing the assembly of the structural frame and the cast polymer leaflets.

With respect to the improved delivery system, the improvements comprise both additional features that can be incorporated into the delivery system to improve performance of valve deployment of prosthetic valves of essentially any design and configuration as well as specific improved designs of the delivery system that advantageously facilitate deployment of the specific prosthetic valve designs described herein using the polymeric leaflet material. Examples include a deflectable member at the distal end of the catheter-based delivery system that locates the native aortic sinus to permit easier visualization of the native annulus and sinus thereby leading to a more accurate and stable deployment in the case of either a self-expanding or balloon expandable system. The deflectable member extends from a portion of the delivery system proximal to the distal and thereof such that the feedback delivered to the user via engagement of the deflectable members of the native annulus guides accurate deployment the prosthetic valve. The deflectable members are radiopaque to be visualized under an imaging modality.

In another embodiment, a three-balloon delivery system is comprised of separate balloons having three expandable members combined with the common inflation system such that these three expandable members or lobes may be independently inflated to selectively engage and interact only with the one of the valve commissures and not interact with the valve leaflets. With this approach, the polymeric valve leaflets are isolated during balloon expansion. This design protects the polymer leaflets and prevents any balloon to leaflet contact that could cause damage to the polymeric leaflets during the balloon-based valve deployment process. In addition to the three-balloon system a delivery system comprising greater than three balloons and including up to six or more balloons is provided to facilitate a round or more circular deployment into the inner diameter of the native annulus. In this embodiment three main inflation balloons are expanded to bring the prosthetic valve in contact with the native annulus and one, two, or three more smaller balloons disposed tangentially at the outer surface of the three main balloons provide for a more circular expansion of the prosthetic valve to engage the native annulus. The three main or primary inflation balloons interact only with the valve commissure shape and do not interact with the leaflets. The secondary, smaller balloons are sized to interact only with the frame located underneath the leaflets and to not contact the polymeric leaflet structure.

In another improvement for the delivery system, the distal end of the delivery catheter has a tear open style sheath that allows for a smaller introducer and easier access to the patient vascular anatomy. When a larger object is pushed through the distal tear-open style sheath, the distal end opens in zip fashion along a perforated line manufactured into a specified length of the distal end of the delivery catheter. The intact (unzipped) sheath has an outer diameter to accommodate the introducer of the valve and delivery system. Once the crimped valve assembly passes through the sheath, the sheath collapses into a smaller profile resulting from the linear reduction in profile along the length of the tear-open region. The tear region can comprise a linear set of openings, or other geometric configuration having perforations or openings in the distal end where progressive tearing at sequential openings that perforate the length of the distal end of the delivery catheter allow the prosthetic valve to be deployed by expansion. Once the tear open process is complete, the overall diameter of the distal end of the delivery system collapses into a profile that is smaller than comparable delivery catheters and allows an improved technique to withdraw the delivery catheter through the length of the patient's vasculature. In every instance of deployment in renewal of a delivery catheter, some inherent trauma is experienced along the walls of the patient's vasculature. A reduced diameter delivery catheter inherently reduces the trauma that occurs upon withdrawal of the delivery system and leads to less traumatic complete removal of the length of the delivery system catheter.

In an additional improvement of a minimally invasive delivery system, a two-balloon delivery system is comprised of a distal balloon that is built into the delivery catheter that is first used for pre-dilation of the native annulus. Once the predilation is complete the distal balloon is deflated and moved forward or distally to allow deployment of a separate proximal balloon that contains the crimped valve in surrounding engagement therewith. In this configuration, the surgeon .may perform both a separate native valve dilation and a subsequent valve deployment procedure with the same delivery apparatus and system. In any of the balloon deployment embodiments described herein, the balloon disposed in conforming engagement with the prosthetic valve may be tapered in the proximal direction relative to the distal and. In such a configuration the distal end of the balloon has a larger distal diameter and a smaller proximal diameter and may be formed in discrete steps from distal to proximal and or may be a smoothly tapered reduction in outer diameter that promotes firm engagement with the distal end of the prosthetic valve relative to the native annulus without exerting pressure or stress on the more proximal portion containing the leaflet assembly.

### BRIEF DESCRIPTION OF THE DRAWINGS

FIGS. 1A and 1B are prosthetic valves having a marker band or structure incorporated into the structural frame for annular orientation and alignment relative to coronary arteries.

FIGS. 2A, 2B and 2C are full frame, scalloped frame, an open frame designs of the structural frame or stent member of the prosthetic valve.

FIG. 2D is a 2-piece construction having a single dip cast leaflet and a separate sealing skirt having inner and outer sealing and where both pieces are configured to engage with each other in a mating relationship and lockable fashion to form an integrated prosthetic valve assembly. The 2-piece construction can be mechanically joined via sutures, rivets, and or welding of the two frames.

FIG. 3 is a design for the structural frame side walls that are sized and shaped to allow the polymer leaflets to collapse when the prosthetic valve is crimped into the collapsed configuration placement in the delivery catheter the aspect ratio A:B of the individual cells is maximized to reduce the strain induced on the polymer. In the right panel, the prosthetic valve is shown in the collapsed configuration to illustrate the advantage of the aspect ratio of the individual cells and the reduced strain induced to the polymer structure.

FIG. 4 is a secondary support structure incorporated into the structural frame support member or stent to create a larger full frame C at the outflow end of the lattice of the frame of the support structure.

FIG. 5 is a self-expanding sealing skirt that opens alongside the balloon expandable frame and functions as a separate sealing skirt overlaying the structure at the inflow end of the support structure at D.

FIG. 6 is shaped sealing skirt at the inflow edge of the prosthetic valve that may have a scalloped form for improved sealing against the native annulus.

FIGS. 7A and 7B are formers used in the manufacturing process to produce a closed cell self-expandable or balloon expandable prosthetic valves showing the positioning of the stent frame on the former during the manufacturing process.

FIG. 8 are leaflets and sidewall masks for the manufacturing embodiment of FIGS. 7A and 7B including locking features, a cavity for frame struts and a fin mask.

FIG. 9 is an outline of a manufacturing process with step-by-step graphics showing a manufacturing process where dip cast leaflets are first created on a former with no frame to form a separate polymer leaflet member of a prosthetic valve assembly when combined with a structural frame. This permits quality intolerance testing of the isolated leaflet structure itself without assembly to the frame.

FIG. 10 is an outline with step-by-step graphics of an alternative manufacturing process of the embodiments of FIG. 9 with the leaflets cured in an alternate configuration.

FIG. 11 is an outline with step-by-step graphics of a manufacturing method that assembles the valve by first electrospinning polymer directly on the frame and joining a cast polymer leaflet to the frame.

FIG. 12 is a two-part balloon catheter having a distal portion to facilitate two-part deployment of the sealing skirt and the structural frame and polymer leaflets.

FIG. 13 is a delivery system with distal probes that provide feedback to the user for positioning of the prosthetic valve at the native annulus.

FIG. 14 a three-lobe or three-balloon design that aligns individual lobes or balloons with the commissure portion of the prosthetic valve to protect the polymer leaflets from damaged during deployment.

FIG. 15 is a schematic of 3 lobe or 3 balloon embodiment having both main and secondary inflation balloons wherein the secondary inflation balloons are located tangentially and longitudinally disposed along main inflation balloons to create alternate deployment strategy and engagement of the prosthetic valve along the length of the balloon members.

FIG. 16 is a tear open style sheath that unzips along a perforated line during valve deployment and yields a reduced diameter profile.

FIG. 17 is a delivery system with a distal balloon built into the catheter that is used for predilation of the native annulus.

### DETAILED DESCRIPTION OF THE INVENTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

The example embodiments described herein relate to improved implantable prosthetic valves, such as prosthetic heart valves having a support structure, stent, or frame coupled with two or more leaflets, and techniques for the manufacture and manufacturability of implantable valves. These embodiments are particularly suited for artificial (not biological tissue) polymeric leaflets, and the resulting artificial valves offer advantages comparable to current approaches with the added benefit of a longer life span. Valves with polymer-based leaflets are advantageous because polymers can offer the same structural support as biological tissue, while being much thinner and allowing the valve to be more easily contracted for delivery. This in turn results in less stress on the polymer as it is contracted which prevents long-term degradation of the valve leaflets. In addition, the manufacturing methods described herein permit fabrication of a valve without suturing or molding leaflets to a support structure or stent, thus promoting high quality repeatable results.

While the embodiments described herein are particularly suited to all for cardiac valves likewise be used with stents and stent graft devices, or other medical devices implantable within the human body.

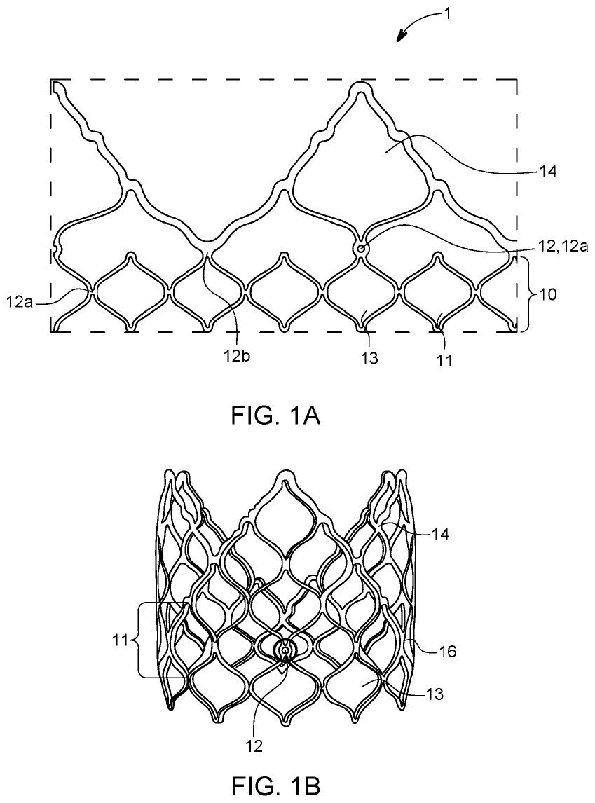

Referring to FIGS. 1A and 1B, a prosthetic cardiac valve 1 incorporates a visible marker band 12 (indicated by arrow in FIG. 1A) as part of the heart valve frame 10 to aid both annular alignment and coronary alignment. In combination with an imaging modality the marker band 12 facilitates accurate placement of the prosthetic valves and gives the user or surgeon improved control over coronary alignment, which in the long term allows for better circulation access to the coronaries and increased blood flow into the coronaries. The marker 12 may be a separate structure disposed in the lattice 11 of the valve frame 10 or may be incorporated into the structure of the stent frame 10 and integral with the stent frame at any point along the frame 10 or lattice 11. The stent frame 10 or lattice 11 may be comprised of cells having multiple sizes such as smaller cells 13 and larger cells 14 with the marker band 12 operably disposed between the differently sized cells in order to assist in visualization and orientation of the valve 1 or stent frame 10.

A plurality of markers 12a, 12b may be placed at points circumferentially around the outer portion of the valve 10 and may be differentiated from each other by size, design structure, or location for orientation around a radius or horizontal axis traversing the inner flow pathway of the valve 1. In one embodiment, a plurality of markers 12a, 12b have separate appearances under an imaging modality and are displaced both vertically and horizontally from each other to allow separate visualization and alignment under an imaging modality The marker will typically be fully encapsulated in polymer. An imaging modality that is capable of permitting the user to view the presence and orientation of the markers is coupled together with the valve delivery system. Together, this combination of the stent frame 10 the leaflets (see below), and the other structural features described herein form an integrated unit for transvascular delivery of the valve 1 together with the ability to orient the valve 1 using the imaging modality to detect and view in real time the presence and orientation of the marker(s). The incorporation of the visibly detectable marker 12 into the stent frame 10 also enables a method wherein the surgeon visualizes the marker bands using the imaging modality and rotates the stent frame 10 of the valve 1 for alignment relative to the native valve annulus and to the coronary arteries of the heart. The method comprises advancing the valve 1 through the vasculature of the patient using a minimally invasive procedure, using the marker(s) to place and orient the valve 1 relative to either or both of the native annulus and the coronary arteries and then deploying the valve to act as a prostatic valve in place of a diseased native valve.

The structure of the individual visible marker 12 may comprise an open cavity in circular, oval, or other configuration to structurally create the visually detectable frame marker 12. When comprising an open cavity, the marker 12 may be directly formed integrally as part of the stent frame 10 for both annular alignment relative to the native annulus and/or for coronary alignment with coronary arteries. As noted previously the stent frame 10 is comprised of a shape memory substance such as a metal or polymer and integral incorporation of the marker band 12 can be readily fabricated using either material. The marker 12 may be preferentially placed on a single side of the stent frame 10 to provide improved visualization and the method of orienting the marker could include orienting one side of the stent frame 10 to a particular position relative to the native annulus or to the coronary arteries and the visualization may occur when the valve 1 and stent frame 10 are in either of the collapsed or expanded configuration.

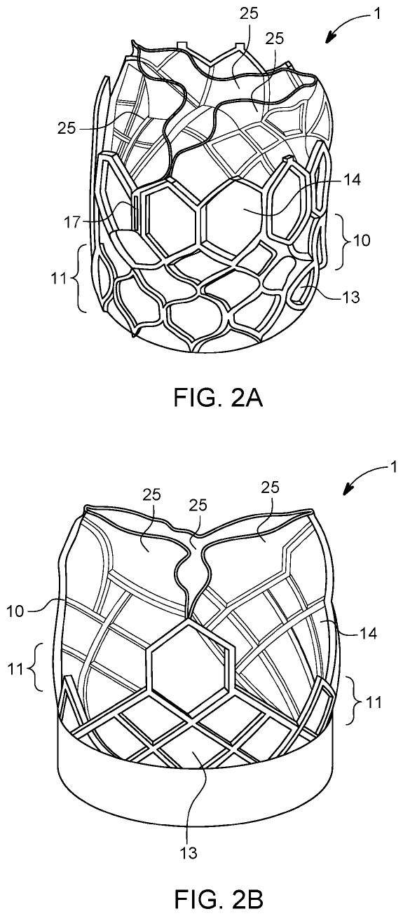

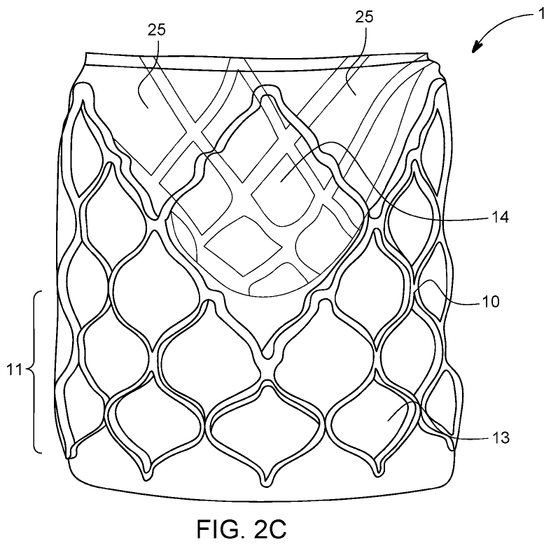

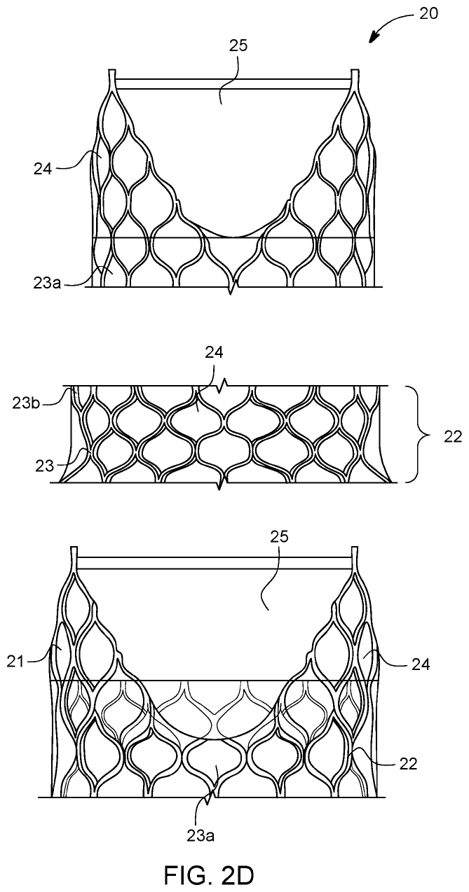

Referring to FIG. 2A-2C, an improvement for heart valve structure is the use of open frame designs to allow for better coronary access. These open frame designs with single strut support structures 17 also keep the leaflets from striking the framework of the heart valve during functioning, which can lead to early failure of the valve implant. The frame design is shaped to provide structural support along the profile of the leaflets. The struts are designed in a manner to follow the curved shape of the leaflets. The frame is left open where the leaflets are not present. This provides an open volume for both blood flow to the coronary sinuses during valve function and ease of catheter access for any post coronary procedures. The open area is reinforced by additional frame structures that may compose of either a single V-shaped strut going across each leaflet body or diamond shaped struts that circumferentially cover the open area. The diamond shaped cells may be larger compared to the cells located at the leaflet sidewalls to allow larger access site for coronary catheters. The frame structure in the open area will not be covered with polymer in order to eliminate the possibility embolizing thin polymer fraying off during valve crimping and expansion.

Referring to FIG. 2D, a multicomponent prosthetic heart valve assembly 20 is comprised of a separate heart valve structure support stent frame, to herein as an inner frame 21 having a size and dimensions as further described below. The inner frame component 21 contains the leaflets and as a separate sealing skirt 23a. The second piece is a separate outer frame 22 that contains a sealing skirt 23b. Both of structures have a stent frame 24 formed from a polymer or shape memory metal for traversing from a collapsed to an expanded configuration as described herein and to separate sealing skirts. The single piece frame 21 contains the full frame docking portion with a subframe containing the leaflets 25. This improvement would allow for a dual frame structure and permitting an overall reduced diameter during placement of the prosthetic heart valve. This allows the inner frame 21 to be crimped onto a delivery system independently of the outer frame 22 that holds the sealing skirt. This independent crimping of both valve structures would reduce the catheter profile by distributing the valve structures axially along the length of the catheter. This independent deployment of the valve structures 21, 22 reduces the outer diameter of the delivery system, thus leading to a smaller delivery profile through the introducer sheath. This construction has a single dip cast leaflet 25 and separate sealing skirts 23a, 23b enables both inner and outer sealing and where both components of the assembly 21, 22 are capable of a mating relationship where the two elements of the assembly 21, 22 are structurally locked together to form the integrated prosthetic valve assembly. The inner frame 21 can include tridents that alight with slots located in the outer frame 22 that self-align during valve expansion. The mechanical locking mechanism can also incorporate a peg-and-hole design that allows a standoff on the inner frame 21 to mate with a similar shaped cavity in the outer frame 22 that aligns and attaches by means of mechanical expansion of the inner frame 21 onto the outer frame 22.

A method of deployment for the two-part valve assembly includes a deployment of the outer, separate sealing skirt frame 22 that is first deployed on the native leaflets. Next the inner frame 21 containing the leaflets is deployed into the separate sealing skirt frame 22. A mating engagement is created by the inner frame 21 including tridents that alight with slots located in the outer frame 22 that self-align during valve expansion. The mechanical locking mechanism can also incorporate a peg-and-hole design that allows a standoff on the inner frame 21 to mate with a similar shaped cavity in the outer frame 22 that aligns and attaches by means of mechanical expansion of the inner frame 21 onto the outer frame 22.

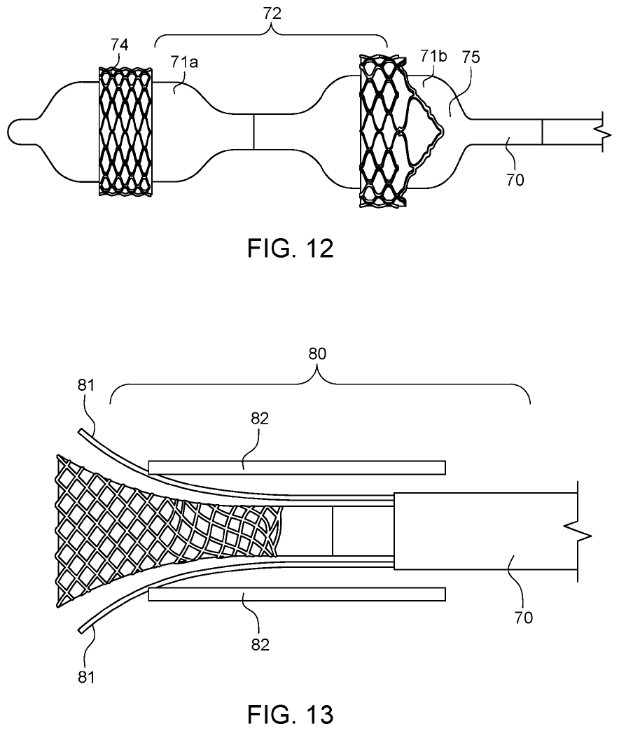

To facilitate this two-part deployment, the delivery catheter may include two balloons (see FIG. 12) where the distal balloon first deploys the outer frame 22, and the proximal balloon deploys the inner frame 21. This allows a single device to both deploy operably deploy the structural features of both sealing skirts and will 23a, 23b into a single unitary structure that provides sealing engagement between both outer skirts and orients the leaflets 25 as part of the overall assembly 20 at the native annulus. The inner frame design 21 design includes features that allow the outer leaflet frame 22 to locate and dock the inner leaflet frame 21. The inner frame 21 can include tridents that alight with slots located in the outer frame 22 that self-align during valve expansion. The mechanical locking mechanism can also incorporate a peg-and-hole design that allows a standoff on the inner frame 21 to mate with a similar shaped cavity in the outer frame 22 that aligns and attaches by means of mechanical expansion of the inner frame 21 onto the outer frame 22.

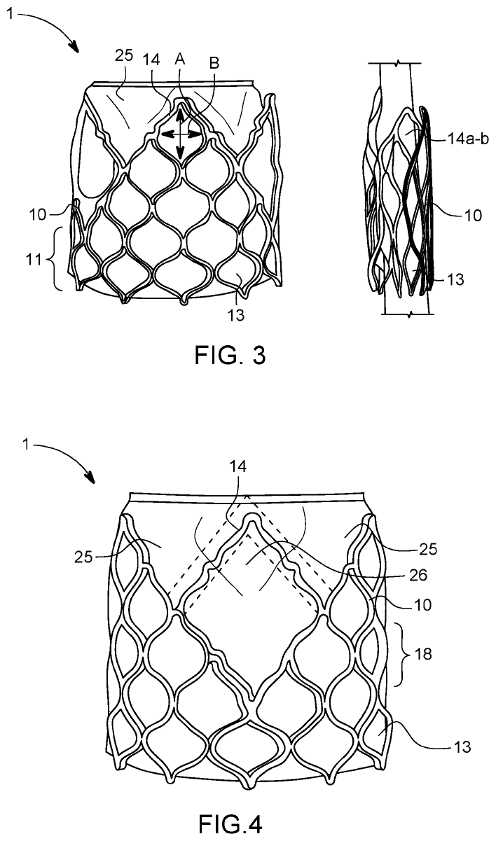

Referring to FIG. 3, the support frame 10 of the prosthetic valve 1 has cells of varying dimensions 13, 14 and the varied cell sizes in the frame valve structure increases the circularity and holding potential at the annulus. As shown in FIG. 3 (see arrow) smaller cells 13 at the bottom (inflow portion) of the heart valve also promote even contact with the native annulus giving better PVL results. The frame sidewall cells 18 are sized to allow the polymer to compress during valve crimping. An optimal linear dimension of a larger cell 14 at the distal or outflow section has a ratio of height to width (1.5-2) defined by dimensions A, B.

Referring to FIG. 4, the aspect ratio of the larger cell 14 to the outflow portion of the valve 1 (height vs. width 1.5 to 2) is maximized to reduce the strain induced on the polymer incorporated into the stent frame 10 when crimping. Larger cells 14 having dimensions D induce a high strain on the polymer which causes the polymer to tear away from the stent frame 10. The aspect ratio 1.5 to 2 between the cell height vs. cell width in the larger cells 14 at the outflow and should be greater than one to reduce the polymer strain during crimping.

The secondary support structure minimizes commissural deflections. Lower commissural deflections are required to reduce the leaflet strains and thereby improve valve durability. The strut geometry allows the stent frame 10 to fold when crimping the valve 1 onto the delivery system (as described below). The geometry of the valve 1 and the secondary support structures are designed to not collapse onto the polymer leaflets 25 during crimping. The frame strut going across the polymer leaflet is designed to bend at the center and remain radially on the outside during the crimping process. The strut 26 provides a secondary purpose of protecting the polymer leaflets 25 from any mechanical damage imparted by the native calcified leaflets. The native calcified leaflets help anchor the TAVR valve, however if the overall height of the leaflets 25 relative to the linear length of the stent frame 10, the leaflets 25 can collide with the stent frame 10 and mechanically damage the valve, leading to a premature valve failure.

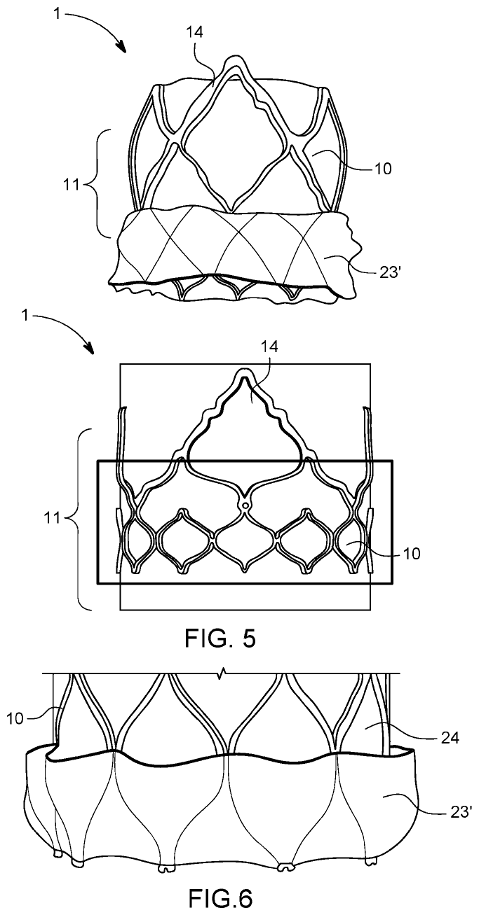

Referring to FIGS. 5 and 6, an improvement for balloon expanded heart valve structure is to add a self-expanding skirt 23′ to the open valve stent frame 10. As shown in FIG. 5, the self-expanding skirt 23′ can be shaped as a tent like structure that tapers from a larger diameter proximally at the inflow and to a smaller diameter distally, closer to but not reaching a midpoint of the overall length of the prosthetic valve 1. The self-expanding sealing skirt is sized to protect the polymer leaflets 25 by providing a barrier between the valve structure and the native calcified leaflets. The constant outward radial force against the annulus will push out the calcified native leaflets and prevent the native leaflets from colliding against the polymer leaflets of the prosthetic valve. In this configuration, the self-expanding skirt 23′ acts as a leaflet protector improving the functionality of the prosthetic heart valve implant 1. The self-expanding skirt 23′ is fabricated and oriented to be crimped down with the expandable stent frame 10 for disposition at the distal end of the minimally invasive delivery catheter.

The self-expanding skirt 23′ can be formed by electrospinning and opens alongside the balloon expandable stent frame 10 and functions as a separate sealing skirt 23′ and polymer leaflet 25 protector. The inner wall of the self-expanding structure of the sealing skirt 23′ is covered with polymer to prevent metal-to-metal interaction between the different metals of the self-expanding skirt 23′ and balloon expandable structures of the stent frame 10.

Referring to FIG. 6, a self-expanding sealing skirt 23′ has a scalloped shaped outflow edge 24 of the sealing skirt 23′ that provides specific attachment points and allows the non-attached portions of the skirt material to balloon out by expanding radially outward for improved engagement with the native annulus and to actively seal the edges around the valve 1 to reduce paravalvular leak. The woven PET sealing skirt 23′ is attached with polymer to the underlying solid polymer inner sealing skirt (not shown). Another advantage provided by the sealing skirt 23′ is the capability of featuring attachment points 28 of the sealing skirt to the valve assembly without sutures, e.g., with polymer. A scalloped shaped outflow edge 24 of the sealing skirt 23′ would provide specific attachment points 28 circumferentially oriented and spaced about the periphery of the external portion of the valve 1.as shown in FIG. 6: This would allow the non-attached skirt material at the scalloped outflow edge 24 to balloon out for acute valve sealing against paravalvular leak (PVL), which would improve function.

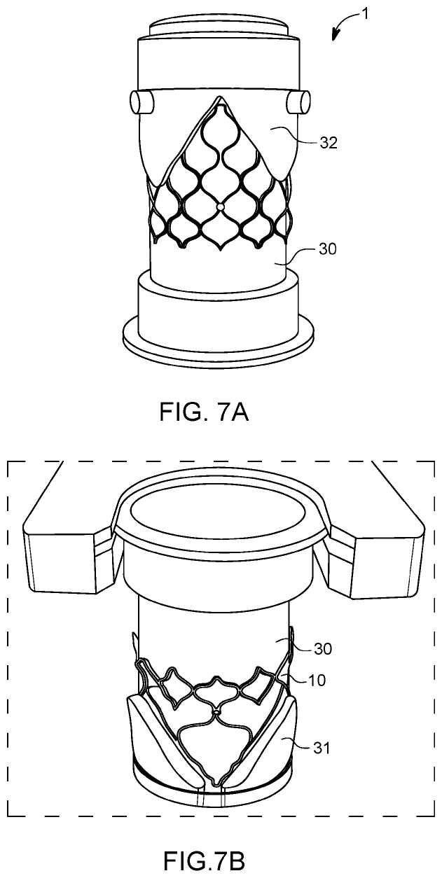

Referring to FIGS. 7A and 7B, FIG. 8 and FIG. 9, the method described herein enables a closed cell self-expandable or balloon expandable heart valve frame 10 to be dip cast in polymer without the need for having an open cell construction. The closed cell construction of the heart valve 1 the frame to remain round during valve crimping and deployment.

Referring to FIG. 7, the elements of the initial construction are the former mold 30 that is devoid of any mechanical attachment or support structure incorporated into the valve 1 prior to forming polymer leaflets 25. The former 30 may be operably mated to a mask 31 that may be superior (FIG. 7A) or inferior (FIG. 7B) to the stent structure and the orientation of the mask and the molder may have either structure at the uppermost portion of the assembly prior to introduction of polymer to form the leaflets 25.

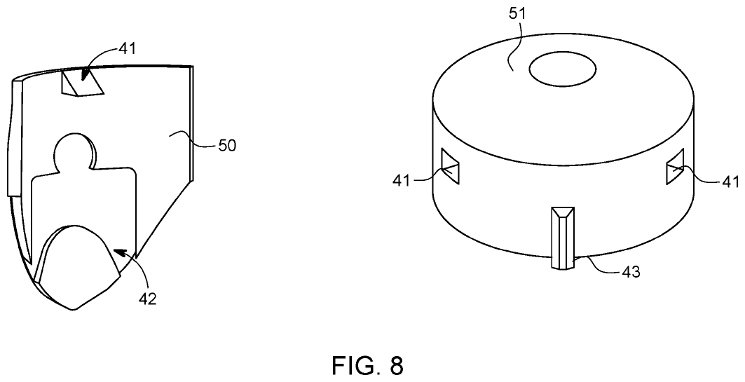

Referring to FIG. 8, an embodiment of the leaflets mask 50, 51 is comprised of locking features 41, leaflet mask former fins leaflets 43, a cavity to receive struts from the stent frame (from the leaflets side) to reside underneath for protection during polymer dipping. In this embodiment, the masks 50, 51 may be mated with the former 30 for the manufacturing embodiment of FIGS. 7A and 7B including locking features, the cavity 41 for frame struts and a fin mask and used in the following techniques to selectively apply polymer to the bare former 30 the assembly of the frame and the pre-cured leaflets 25 and especially for the dipping of the assembly of the stent frame 10 and the former 30 prior to being placed in a humidity chamber for curing of the polymer. Specially designed leaflet masks 51, 52 are preferably applied to the stent frame 10 frame and polymer leaflet 25 assembly. The use of the masks 51, 52 inhibits or prevents polymer ingress onto the cured leaflets 25 or polymer coverage over cells of the stent 10 that are meant to stay open for coronary access post valve implantation. The masks 51, 52 may include a silicone gasket (not shown) circumferentially arrayed around the outer edge diameter of the masks to seal against polymer ingress during the polymer dipping process 114, 123.

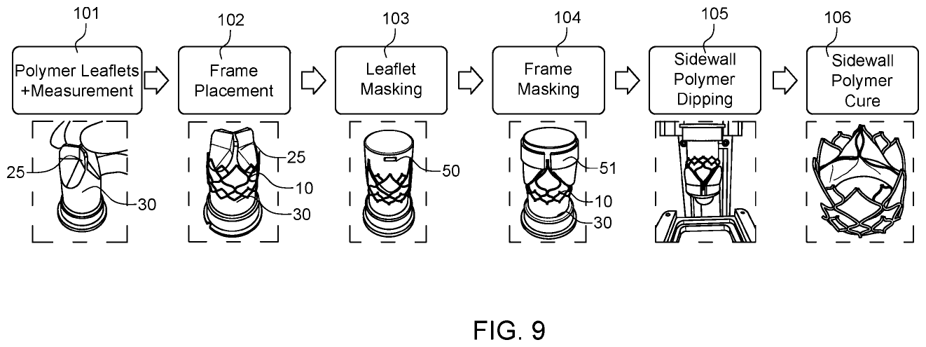

Referring to FIG. 9, the manufacturing process steps include the following methods as follows and the structures and assemblies are produced as follows:
1. Dip cast leaflets 25 are formed on a new, bare former 30, with no stent frame 10 present. At method step 101, the polymer leaflets are formed, measured, and verified to be within critical tolerances and dimensions for assembly into the final prosthetic valve. Once quality control measurements are deemed to have been satisfied, the frame is placed on the polymer leaflets still integrated with the bare former 30 at the next method step. Subsequently, in the leaflet masking method step 103, the leaflets 25 are masked with a protective cover 50 prior to curing of the leaflets 25. A frame mask 51 is added to the assembly as shown in FIG. 9 as part of a frame masking step 104 to make a complete assembly prior to application of liquid polymer. The specially designed leaflet 50 and frame masks (see FIG. 8) onto the frame and leaflet assembly. This prevents any polymer ingress onto the cured leaflets or polymer coverage over frame cells that are meant to stay open for coronary access post valve implantation. Once masked, the assembly is dipped in polymer with the leaflets 25 facing down. The polymer is allowed to flow around the unmasked struts of the frame 10 to bond the leaflets 25 to the frame assembly 10. The leaflet/frame 50, 51 mask may include a silicone gasket (not shown) at the edge to seal against polymer ingress.

Next, a polymer dipping step 105 integrates the previously formed leaflets 25, the stent frame, 10 into an integrated assembly with selective frame 10 and leaflet 25 portions of the overall valve 1 protected from exposure to liquid polymer. The former 30 is dipped with the leaflets 25 facing down. Following the dipping step 105 the assembly is placed in a humidity chamber for a specified time under specified humidity condition for polymer thickening and next place the leaflets and in an oven for a specified time to fully cure the leaflets 25—see US Publ. 20190060061A1 for curing of the dipped polymer. Following curing in the humidity chamber the frame masks 51 are removed and the prosthetic valve 1 is placed in an oven to fully cure the assembly. The polymer leaflets may be trimmed using either an ultrasonic knife, die cutting, or laser cutting system.

The self-expanding or balloon expandable stent frame 10 is secured over the cured leaflets 25. Once curing is complete, leaflet and frame masking 50, 51 are removed and the valve 1 inspected for dimensions, tolerances and any necessary quality control parameter.

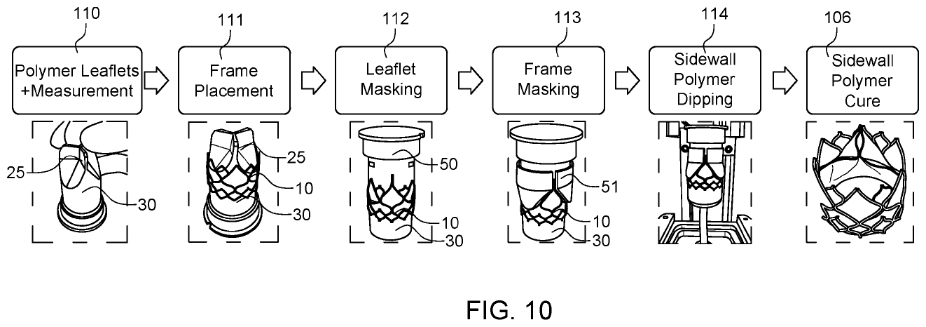

In another embodiment of the manufacturing process, referring to FIG. 10, the several steps analogous to the methodology of FIG. 9 are performed. Once the assembly of the stent frame in polymer leaflets are dipped in polymer, the frame masks are removed from the humidification chamber and cured in an oven with the leaflets facing up. This embodiment of the valve assembly process allows the premade leaflets 25 to cure with the leaflet tips (not shown) facing up, which prevents polymer ingress over the leaflet commissure. The methodology of FIG. 10 allows permits less polymer use—polymer usage is reduced when the valve is dipped with the leaflets facing up because in this orientation the leaflet mask 51 is not dipped in polymer. In general, the lower the surface area of the parts of the assembly of the former 30, the stent frame 10, and the masks 51, 52 being dipped in polymer, the less polymer is extracted from the polymer well during dipping. The former 30 includes a thread at the base of the former to allow the attachment of a holder that allows the assembly to be placed in the oven with the leaflets facing down for final leaflet curing. This also allows the former 30 to be held and dipped in two orientations. Accordingly, the assembly of the former 30, the stent 10, and the masks 51, 52 may be dipped in polymer and then cured with the leaflets facing either in a down configuration (FIG. 9) are facing up (FIG. 10) or a combination of both. In particular, the methodology of FIG. 10 may be performed to reduce the amount of total polymer being used in the methodology 110 to 115 for a portion of the curing process enough to secure the advantage of decreased polymer usage and then the orientation changed for any suitable further curing process to complete the assembly process. As noted above, either process may involve the step of trimming the polymer leaflets 25 using either an ultrasonic knife, die cutting, or laser cutting system.

With respect to the structures and method steps described above, the formation of the final assembly comprising the polymer leaflets 25 and stent frame 20 is designed for either deployment by self-expansion upon placement of the native cardiac annulus or by deployment of a conventional or improved balloon deployment system as described below.

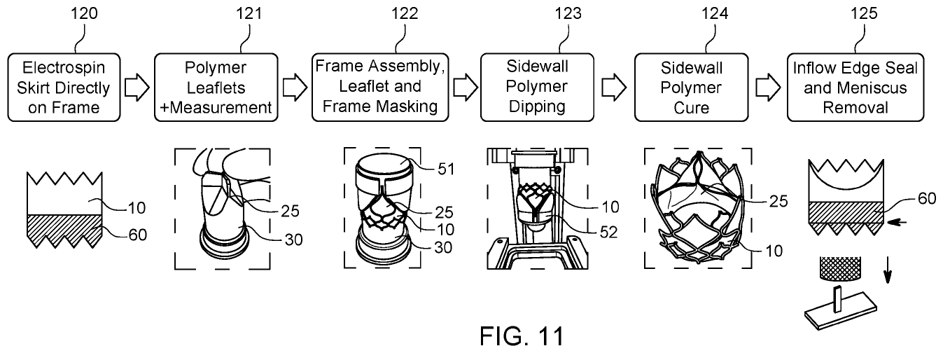

Referring to FIG. 11, another embodiment for a method of assembling the valve by electrospinning a sealing skirt 60 directly on the frame and joining cast polymer leaflets 25 to the stent frame 10. In the first step 120 the sealing skirt 60 is electricity on directly onto the stent frame 10. In the next step 121 the leaflets 25 are formed by dipping the bare former 30 in polymer. Once the polymer leaflets 25 has been formed, this step 121 includes trimming and performing quality analysis of several parameters of the polymer leaflets including dimensions (height, thickness), overall quality, and competency of the downstream ends of the valvular portion of the overall polymer leaflets structure. In the next step, the leaflet mask 51, the polymer leaflets 25 and the stent frame 10 to form an assembly and the outflow edge seal is formed. When electrospinning the sealing skirt 60, an inner layer of polymer is first cured on the valve 1 prior to spinning. This provides a cured polymer substrate for the electrospun micron sized polymer fibers to attach during the electrospinning process. The prosthetic valve 1 production is complete by sealing the inflow edge at the base of the stent frame 10 that is attached to the inner cast polymer layer (not shown) that is assembled with both the stent frame 10 and the sealing skirt 60. In this embodiment, as in the methods described in FIGS. 9 and 10, the leaflets 25 are formed using the bare former 30. The leaflet and sidewall masks 51, 52 are assembled over pre-cured leaflets 25 and the frame sidewall is dipped in polymer to integrally form the connection between the stent frame 10 and the sidewall of the leaflet structure and to form the outflow edge seal.

Referring to FIG. 12, to facilitate the two-part deployment of the two-piece valve of for example FIG. 2D, a delivery catheter 70 has two balloons 71a, 71b disposed at the distal end of the catheter. The most distal balloon 71a first deploys the separate sealing skirt frame 74 having a first frame element, and the more proximal balloon 71b deploys the separate leaflet frame 75 having the second frame element integral with the polymer leaflets. In this configuration, a single delivery device assembly 72 deploys both the sealing skirt element 74 and the separate combination of the stent frame 10 and polymeric leaflets 25 element. Either or both of the sealing skirt element 74 and the polymer leaflet element 75 contain features that allow the polymeric leaflet component 75 to engage and integrally lock with each other to form a single integrated assembly 72 that functions as the prosthetic valve 1 once integrated.

Referring to FIG. 13, an improvement to a delivery system 80 for a heart valve replacement procedure uses deployable sensors 82 or “feelers” that provide mechanical feedback to the surgeon regarding the position and orientation of the distal end of the delivery system 80. In a preferred embodiment the feelers are disposed longitudinally parallel to the length of the distal end of the catheter 70. The feelers 81 may be maintained at the distal end by the outer body of the catheter 70 or maybe enclosed within an annular capsule 82 that surrounds the portion of the distal end of the catheter 70 containing the feelers 81. The feelers 81 are deployed by either advancing the distal end of the catheter 70 relative to the capsule 82 or withdrawing the capsule 82 proximally to allow the feelers 81 to deploy outward and preferably circumferentially at points around the prosthetic valve 1. Manual feedback to the surgeon from the feelers 81 is provided by a mechanical connection such as a guidewire or other mechanical rigid member running the axial length of the catheter to connect the mechanical sensors to sense the position and orientation thereof at the proximal end of the catheter. The manual feedback received by the user/surgeon at the proximal end enables a more sensitive and stable deployment of the prosthetic valve 1 in either a self-expanding or balloon-expandable system.

As is reflected in the deployment of the feelers 81 in FIG. 13, a method of the invention includes the use of the delivery system 80 having the mechanical or structural addition of the feelers 81 to the distal end of the system and using this mechanical feedback provided through the axial length of the catheter to the proximal end of the delivery system in a method to deploy the prosthetic valve 1 such that mechanical feedback informs the surgeon of when to deploy the prosthetic valve either by the mechanical feedback of the feelers 81 or through traditional imaging techniques or a combination of both. Thus, the addition of feelers to the distal end of the delivery would enables selective positioning of the delivery system 80 prior to deployment of the prosthetic valve 1 and as a precursor step to final positioning of the prosthetic valve 1 during deployment.

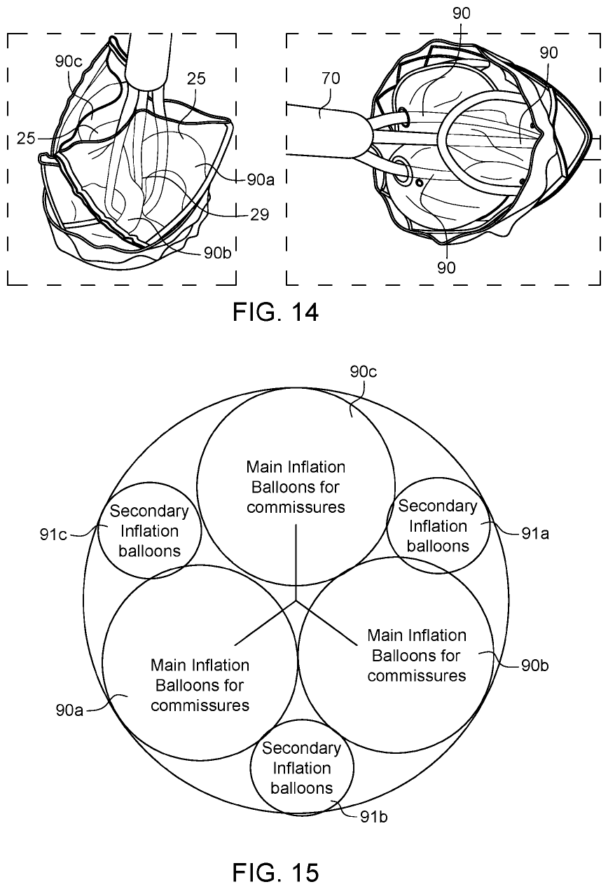

Referring to FIGS. 14 and 15, a modified design for the deployment strategy of the prosthetic valve 1 uses a combination of balloons to enable a more complete and predictable deployment and expansion of the prosthetic valve 1. The modified balloon configuration is comprised of three main inflation balloons 90a, 90b, 90c or lobes that interact only with the shape of the commissures 90 of the prosthetic valve 1. In this configuration, the leaflets 25 are separated from the main inflation balloons 90a-c so that no contact is created between the balloons 90a-c and the leaflets 25 during expansion. The design protects the polymer leaflets 25 and prevents any balloon-to-leaflet interaction that may cause leaflet damage during the valve deployment process. Preferably, the tri-shaped balloon design and orientation matches the shape of the heart valve commissure 90.

Referring to FIG. 15, a cross sectional schematic shows the orientation of the main inflation balloons 90a-c for orientation along the valve commissures 90. In one embodiment, secondary inflation balloons 91a-c are disposed between each of the main inflation balloons 90a-c and are positioned to engage longitudinally and tangentially along the length of the main balloons. The main and secondary balloons can be inflated separately or individually or in series such that the main inflation balloons are all inflated first and the secondary inflation balloons are inflated second. Accordingly, each of the six balloons can have a dedicated lumen or separate lumens can be provided to inflate any of the six balloons selectively, in groups of three, or collectively in stages. Preferably, a selective pressure valve is incorporated into the delivery system so that once a certain pressure is reached, the pressure valve alternates from inflating the main balloons 90a-c to inflating the secondary balloons 91a-c. In this methodology, and more consistently round deployment is created by outward pressure on the prosthetic valve first by the main inflation balloons 90a-c contacting the commissures and second by deployment of the secondary inflation balloons 91a-c. In the fully deployed configuration, The inner secondary balloons 91a-c fill the voids between the larger main expansion balloons 90a-c.

As noted, the three main balloons 90a-c interact only with the valve commissures 90 and do not interact with the leaflets 25. The smaller balloons 91a-c are sized to only interact with the frame located underneath the leaflets 25 and do not contact the leaflets 25. The smaller secondary balloons 91a-c would promote the even deployment of the stent frame 10 during prosthetic valve 1 deployment.

A method for use includes separate steps of inflating the main and secondary inflation balloons together with a discrete set of pressurization times and pressure values imposed selectively to the different balloon types. The method step also includes a separate discrete inflation phase where the leaflets are not in contact with the main inflating balloons.

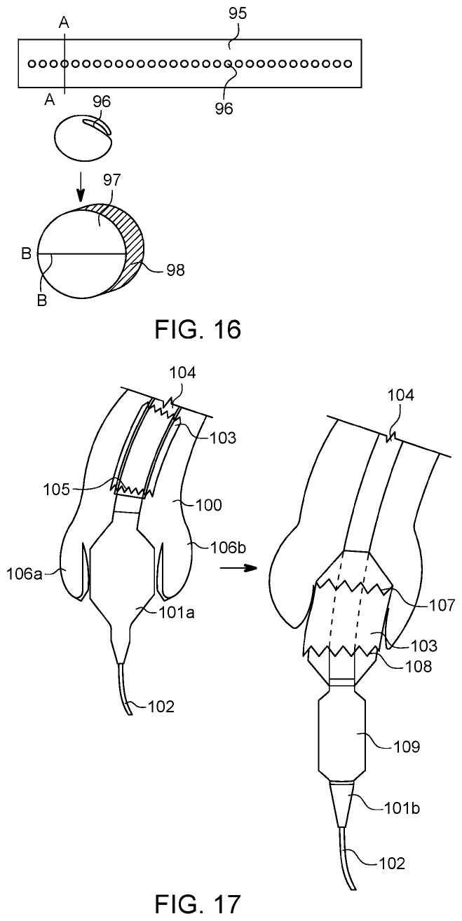

Referring to FIG. 16, a tear open style sheath 95 allows for a smaller introducer diameter at the distal end of the catheter 70 and a smaller introducer diameter results in easier access to the patient anatomy. Once the surgeon advances a structure that is larger than the internal diameter of the tear open sheath 95, including the prosthetic valve 1, the larger diameter of the prosthetic valve 1 zips open the distal end of the tear open sheath 95 along the linear set of perforations 96. The unzipped sheath having openings 96 along axis A increases in outer diameter B to accommodate the introduction of the prosthetic valve 1 and delivery system 70. Once the crimped, the valve assembly passes through the tear open sheath 95, the sheath collapses into a smaller diameter profile and can more easily be withdrawn through the patient's vasculature. Accordingly, the invention includes both a structure and a methodology to deploy a tear open style sheath 95 that has an initial diameter smaller than the diameter of the integrated leaflet/stent frame assembly, expansion to an outer diameter that causes the tear-open sheath 95 to separate along a linear axis of perforations 96 followed by collapse of the tear open sheath 95 to a much smaller diameter once the prosthetic valve 1 is deployed at the native annulus The zipper-style tear open sheath 95 allows the manufacture of a smaller diameter introducer and easier access during the advancement heart valve delivery system to the native annulus to deploy the prosthetic valve 1 and then removal of a structure having a much smaller outer diameter.

Referring to FIG. 17, a distal balloon 101a is built into a catheter 100 that is especially designed to accommodate the passage of the distal balloon 101a and has lateral expandable features at the distal tip thereof 106a 106b that are used for balloon pre-dilation of the native annulus prior to deployment of the prosthetic valve 1. The lateral expandable regions 106a, 106b may be inflatable or expandable through mechanical action or may be static relative to the axial structure of the catheter but constructed to be susceptible to be actively forced in a substantially annular fashion against the native annulus by the expansion of the distal balloon 101a such as by lateral openings that make these regions 106a, 106b more readily susceptible to be disposed perpendicular to the linear pathway of the catheter. The positioning of the distal balloon 101a is preferably just proximal to the distal tip of the catheter 102 and just proximal of the distal portion of the prosthetic valve 105 with the proximal portion of the prosthetic valve 103 also disposed around the body of the catheter 100. Interior to the catheter 100 and running proximally to the most proximal portion of the valve 103 may be any number of features generally described at 104 that pass through the interior of catheter 101 to facilitate outward expansion of distal balloon 101a. Once pre-dilation is complete by expansion of distal balloon 101a, the distal balloon is deflated into a reduced diameter 109 and moved distally to permit advancement of a proximal valve deployment balloon 103. Thus, the methodology of the invention allows the surgeon to perform both the pre-dilation and valve deployment functions using the same catheter device, reducing procedural time and complexity. Procedurally, the catheter 100 bearing the distal balloon 101a is advanced to the native annulus with the balloon in a contracted position. Once a securely located within the native annulus the distal balloon 101a is expanded causing regions 106a, 106b to exert outward force, substantially annular, and substantially perpendicular to the longitudinal axis of the catheter 100, on the native annulus to produce a dilated native annulus in preparation for deployment of the prosthetic valve. Then, the distal balloon 101a is deflated and the catheter 100 advanced distally until the prosthetic valve is in position for deployment at the native annulus. In this configuration the most proximal portion of the valve 107 and the most distal portion of the valve 108 are located across a pre-dilated native annulus. Following deployment of the prosthetic valve, both the distal balloon 101a and the proximal valve deployment balloon 103 are deflated and the entire device withdrawn through the vasculature.

In some embodiments, the electrospun polymer and the polymeric valvular body are both a siloxane polyurethane urea. In some embodiments, the polymer of the valvular body can be a siloxane polyurethane urea. In all the aforementioned embodiments pertaining to a siloxane polyurethane urea, that siloxane polyurethane urea can include: a first, a second, a third, and a fourth segment. The first segment can have a structure of -A¹-L¹-A¹-, where L¹ is the residue of a first diisocyanate. A¹ is the residue of a poly(C₁-C₁₂alkane diol). The second segment can have the residue of a first siloxane-containing diol. The third segment can have the residue of a second siloxane-containing diol, and the fourth segment can have the residue of a C₁-C₁₂alkane diamine, where the segments are each covalently bonded to each other through the residue of a diisocyanate.

In all the aforementioned embodiments pertaining to a siloxane polyurethane urea, that siloxane polyurethane urea can have a structure of A⁴-L⁴-A³-L³-A²-L²-A¹-L¹-A¹-L²-A²-L³-A³-L⁴-A⁴, where L¹ can be the residue of a first diisocyanate. A¹ can be the residue of a poly(C₁-C₁₂alkane diol). L² can be the residue of a second diisocyanate. A² can be selected from -A¹-L¹-A¹-, the residue of a first siloxane-containing diol, the residue of a second siloxane containing diol, and the residue of a C₁-C₁₂alkane diamine. L³ can be the residue of a third diisocyanate. A³ can be selected from the residue of a first siloxane-containing diol, the residue of a second siloxane containing diol, and the residue of a C₁-C₁₂alkane diamine. L⁴ can be the residue of a fourth diisocyanate. A⁴ can be selected from -A¹-L¹-A¹-, the residue of a first siloxane-containing diol, the residue of a second siloxane containing diol, and the residue of a C₁-C₁₂alkane diamine. In the structure of A⁴-L⁴-A³-L³-A²-L²-A¹-L¹-A¹-L²-A²-L³-A³-L⁴-A⁴, at least one instance of A², A³, or A⁴ can be the residue of a second siloxane containing diol; and at least one instance of A², A³, or A⁴ can be the residue of a C₁-C₁₂alkane diamine.

In all the aforementioned embodiments pertaining to a siloxane polyurethane urea, that siloxane polyurethane urea can include: a first structure of A A²-L³-A³-L⁴-A⁴ (Formula II), and/or a second structure of A⁴-L⁴-A²-L³-A³-L²-A¹-L¹-A¹-L²-A³-L³-A²-L⁴-A⁴ (Formula III). For the first and second structures:
L¹ can be the residue of MDI;
A¹ can be the residue of PHMO;
L² can be the residue of MDI;
A² can be the residue of PDMS;
L³ can be the residue of MDI;
A³ can be the residue of BHTD;
L⁴ can be the residue of MDI; and
A⁴ can be the residue of EDA.

In many example embodiments, an implantable valve is provided that includes: a frame including a plurality of deflectable struts; and a polymeric valvular body coupled with the frame, the polymeric valve body including a plurality of artificial leaflets, where the implantable valve has a radial dimension and is capable of transitioning between a contracted state and an expanded state, where the radial dimension is relatively smaller in the contracted state than in the expanded state.

In these valve embodiments, the frame and valvular body can be coupled together with cured polymer. The frame can be encapsulated in the cured polymer. The polymeric valvular body can be composed of the cured polymer.

In these valve embodiments, the implantable valve can have a longitudinal axis and, when the implantable valve is in the expanded state, the plurality of deflectable struts are transverse to the longitudinal axis. When in a fully contracted state, the plurality of deflectable struts can be parallel or substantially parallel to the longitudinal axis. The valve can further include a plurality of longitudinal struts, each of the plurality of longitudinal struts positioned at a commissure between adjacent leaflets. Each of the plurality of longitudinal struts can be parallel to a longitudinal axis of the implantable valve when the implantable valve is in the expanded and contracted configurations. The plurality of deflectable struts can cross and form a plurality of cells. The frame can include a first row of cells located adjacent a downstream end of the frame, where the plurality of longitudinal struts are in the first row of cells. The frame can include a second row of cells located upstream of the first row of cells, where no longitudinal strut is in the second row of cells.

In many example embodiments, methods of implanting a prosthetic valve are provided, where the methods include: moving the prosthetic valve, with an elongate delivery device while the prosthetic valve is in a contracted state, through a body of a recipient; and implanting the prosthetic valve in the body of the recipient by, at least, deploying the prosthetic valve from the delivery device, where the prosthetic valve is implanted in an expanded configuration, and where the prosthetic valve is in accordance with any of the aforementioned valve embodiments.

The invention includes A prosthetic heart valve comprising:
a polymeric leaflet structure integrally formed with a stent frame;
a visibly detectable marker integrated into the stent frame formed from a lattice of cells.

A prosthetic heart valve wherein the visibly detectable marker is comprised of a feature containing an opening and is incorporated into the stent frame at a juncture between cells of the lattice.

A prosthetic valve, wherein a plurality the visibly detectable markers is arrayed along a horizontal axis of the prosthetic valve.

An assembly comprising:
a bare former and a set of polymer leaflets wherein the set of polymer leaflets are cured thereon in the absence of the stent frame.

An assembly comprising:
a former, a set of polymer leaflets, a leaflet mask and a frame mask wherein the leaflet mask is disposed proximate three commissures of a prosthetic heart valve.

A prosthetic heart valve comprised of:
a 2-piece construction of:
dip cast polymer leaflets integral with a first frame and
a separate sealing skirt integral with a second frame having inner and outer seals and where both the polymer leaflets and the sealing skirt are configured to engage in a mating and lockable relationship to form an integrated prosthetic valve assembly.

A prosthetic valve wherein the first frame and the second frame of the 2-piece construction are mechanically joined by sutures, rivets, and welding and combinations thereof.

A method to manufacture a prosthetic heart valve comprising:
forming polymer leaflets on a bare former;
placing a stent frame on the combination of the polymer leaflets and the bare former;
masking the leaflets and frame to form an integrated assembly of a mask, the former and the polymer leaflets;
dipping the integrated assembly in polymer;
curing the polymer encapsulating the integrated assembly.

A method further comprising measuring dimensions of the polymer leaflets on the bare former prior to placement of the stent frame.

A method further comprising electrospinning a sealing skirt on the stent frame.

A method of claim further comprising aligning a cavity in the mask with struts of the stent frame.

A method further comprising disposing a gasket circumferentially around the outer edge diameter of the masks to seal against polymer ingress during the dipping step.

A prosthetic valve delivery system comprising:
a two-part balloon catheter having a proximal balloon and a distal balloon, a sealing skirt comprising a first stent frame element surrounding the distal balloon and second stent frame element integral with polymer leaflets and surrounding the proximal balloon.

A delivery system for a prosthetic heart valve comprising:
a catheter having a distal and containing a prosthetic valve in a collapsed configuration; deployable mechanical sensors; a mechanical connection running the entire axial length of the catheter to provide mechanical feedback to a user for positioning and orientation of the prosthetic valve at the distal end of the catheter.

A delivery system further comprising a capsule containing the mechanical sensors.

A delivery system for a balloon-expandable prosthetic heart valve comprising main and secondary inflation balloons wherein the main inflation balloons engage the commissures of the prosthetic valve along a length thereof.

A delivery system wherein secondary inflation balloons are located tangentially and longitudinally disposed along the main inflation balloons and along a length thereof.

A delivery system further comprising a check valve for staged inflation of the main and secondary balloons.

A prosthetic valve delivery system comprising a catheter having a tear-open sheath at a distal end thereof and having a set of perforations along the tear-open portion.

A catheter wherein the perforations are aligned along a linear axis at the distal end of the catheter and wherein an outer diameter of the prosthetic valve is larger than the inner diameter of the tear-open portion.

A prosthetic valve delivery system comprising: a catheter to accommodate the passage of a distal balloon and having has lateral expandable regions at the distal tip thereof, wherein the lateral expandable regions are inflatable or expandable through mechanical action exerted by the distal balloon to force the expandable regions against the native annulus by the expansion of the distal balloon and a proximal valve deployment balloon having the prosthetic valve in conforming engagement therewith.

As used herein and in the appended claims, the singular forms “a”, “an”, and “the” include plural referents unless the context clearly dictates otherwise.

Where a range of values is provided, each intervening value, to the tenth of the unit of the lower limit unless the context clearly dictates otherwise, between the upper and lower limit of that range and any other stated or intervening value in that stated range, is encompassed within the disclosure and can be claimed as a sole value or as a smaller range. Where the stated range includes one or both of the limits, ranges excluding either or both of those included limits are also included in the disclosure.

Where a discrete value or range of values is provided, that value or range of values may be claimed more broadly than as a discrete number or range of numbers, unless indicated otherwise. For example, each value or range of values provided herein may be claimed as an approximation and this paragraph serves as antecedent basis and written support for the introduction of claims, at any time, that recite each such value or range of values as “approximately” that value, “approximately” that range of values, “about” that value, and/or “about” that range of values. Conversely, if a value or range of values is stated as an approximation or generalization, e.g., approximately X or about X, then that value or range of values can be claimed discretely without using such a broadening term.

However, in no way should this specification be interpreted as implying that the subject matter disclosed herein is limited to a particular value or range of values absent explicit recitation of that value or range of values in the claims. Values and ranges of values are provided herein merely as examples.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

We claim:

1. A prosthetic heart valve comprising:
a polymeric leaflet structure integrally formed with a stent frame;
a visibly detectable marker integrated into the stent frame formed from a lattice of cells.

2. The prosthetic heart valve of claim 1, wherein the visibly detectable marker is comprised of a feature containing an opening and is incorporated into the stent frame at a juncture between cells of the lattice.

3. The prosthetic heart valve of claim 1, wherein a plurality of the visibly detectable markers are arrayed along a horizontal axis of the prosthetic valve.

4. An assembly comprising:
a bare former and a set of polymer leaflets wherein the set of polymer leaflets are cured thereon in the absence of the stent frame.

5. An assembly comprising:
a former, a set of polymer leaflets, a leaflet mask and a frame mask wherein the leaflet mask is disposed proximate three commissures of a prosthetic heart valve.

6. A prosthetic heart valve comprised of:
a 2-piece construction of:
(a) dip cast polymer leaflets integral with a first frame and
(b) a separate sealing skirt integral with a second frame having inner and outer seals and where both the polymer leaflets and the sealing skirt are configured to engage in a mating and lockable relationship to form an integrated prosthetic valve assembly.

7. The prosthetic valve of claim 6, wherein the first frame and the second frame of the 2-piece construction are mechanically joined by sutures, rivets, and welding and combinations thereof.

8. A method to manufacture a prosthetic heart valve comprising:
(1) forming polymer leaflets on a bare former;
(2) placing a stent frame on the combination of the polymer leaflets and the bare former;
(3) masking the leaflets and frame to form an integrated assembly of a mask, the former and the polymer leaflets;
(4) dipping the integrated assembly in polymer;
(5) curing the polymer encapsulating the integrated assembly.

9. The method of claim 8, further comprising measuring dimensions of the polymer leaflets on the bare former prior to placement of the stent frame.

10. The method of claim 8, further comprising electrospinning a sealing skirt on the stent frame.

11. The method of claim 8, further comprising aligning a cavity in the mask with struts of the stent frame.

12. The method of claim 8 further comprising disposing a gasket circumferentially around the outer edge diameter of the masks to seal against polymer ingress during the dipping step.

13. A prosthetic valve delivery system comprising:
a two-part balloon catheter having a proximal balloon and a distal balloon, a sealing skirt comprising a first stent frame element surrounding the distal balloon and second stent frame element integral with polymer leaflets and surrounding the proximal balloon.

14. A delivery system for a prosthetic heart valve comprising:
a catheter having a distal and containing a prosthetic valve in a collapsed configuration; deployable mechanical sensors; a mechanical connection running the entire axial length of the catheter to provide mechanical feedback to a user for positioning and orientation of the prosthetic valve at the distal end of the catheter.

15. The delivery system of claim 14, further comprising a capsule containing the mechanical sensors.

16. A delivery system for a balloon-expandable prosthetic heart valve comprising main and secondary inflation balloons wherein the main inflation balloons engage the commissures of the prosthetic valve along a length thereof.

17. The delivery system of claim 16, wherein secondary inflation balloons are located tangentially and longitudinally disposed along the main inflation balloons and along a length thereof.

18. The delivery system of claim 16, further comprising a check valve for staged inflation of the main and secondary balloons.

19. A prosthetic valve delivery system comprising a catheter having a tear-open sheath at a distal end thereof and having a set of perforations along the tear-open portion.

20. The catheter of claim 19, wherein the perforations are aligned along a linear axis at the distal end of the catheter and wherein an outer diameter of the prosthetic valve is larger than the inner diameter of the tear-open portion.

21. A prosthetic valve delivery system comprising: a catheter to accommodate the passage of a distal balloon and having has lateral expandable regions at the distal tip thereof, wherein the lateral expandable regions are inflatable or expandable through mechanical action exerted by the distal balloon to force the expandable regions against the native annulus by the expansion of the distal balloon and a proximal valve deployment balloon having the prosthetic valve in conforming engagement therewith.
